(19) United States
(12) Patent Application Publication
(10) Pub. No.: US 2009/0029347 A1
(43) Pub. Date: Jan. 29, 2009

(54) Method for Identifying Multiple Analytes Using Flow Cytometry

(76) Inventor: Jerry T. Thornthwaite, Henderson, TN (US)

Correspondence Address: LANIER FORD SHAVER & PAYNE P.C., P O BOX 2087, HUNTSVILLE, AL, 35804-2087, US

(21) Appl. No.: 11/829,877

(22) Filed: Jul. 27, 2007

(51) Int. Cl. C12Q 1/68; C12Q 1/70; G01N 33/53

(52) U.S. Cl. 435/5; 435/6; 435/7.1

(57) ABSTRACT

The present disclosure is directed to a method of using antibodies attached to different types of microspheres against different antigens located within a biological sample. Optical and electronic particle detection may be used to separate the microspheres via flow cytometry, allowing the subsequent measurement of multiple analytes in a single sample of body fluid by separating and gating such analytes based on the type of microsphere to which the analyte is coupled. According to the present disclosure, various biological components may be attached to microspheres of different volumes, shapes, conductivity, densities, and/or colors to detect biological components by gating on the type of microsphere and analyzing the biological component attached thereto.

### FIELD OF THE DISCLOSURE

The present disclosure relates generally to laboratory diagnostics as related to the use of microspheres in flow cytometry. More particularly, the present disclosure relates to the use of electronic particle volume to differentiate by volume different microspheres in order to separate and identify analytes attached thereto.

### BACKGROUND OF THE DISCLOSURE

Current advances in science and medicine have heightened the importance of the analysis of clinical specimens. A wide variety of assays are known in the art to determine qualitative and/or quantitative characteristics of a specimen. Detection of multiple analytes through single step assay processes is very limited and provides inconsistent results due to low sensitivity and the inherent limitations of certain reagents. There is an important need to quickly and accurately analyze clinical and research serum samples using a minimum quantity of blood by a simple, common analysis platform. Accordingly, a multiplexing assay is needed to perform simultaneous multiple determinations of analytes within a single biological sample.

One technique for accounting, examining, and sorting multiple analytes occurring in a biological sample involves flow cytometry. Flow cytometry allows simultaneous multiparametric analysis of physical and/or chemical characteristics of particles flowing through optical and/or electronic detection apparatuses. Current optical detection systems monitor changes in absorbance, light scatter, and fluorescence. Sensitivity of these systems, however, is limited by a variety of factors such as sample fluid flow and sensor placement. Electronic detection is achieved by suspending particles in conducting fluid, then passing them through a small aperture or orifice. An electronic field is applied across the aperture or orifice, creating a current. When a particle passes through the aperture, the resistance across the orifice increases. The increase in resistance at constant current results in an increase in voltage across the orifice, which is directly related to the volume of the particle. The measurable voltage pulse is generated which can be analyzed and used to conduct further operations such as conductivity, resistivity, capacitance and shape modeling of the particle.

In both optical and electrical detection systems, microspheres are useful analytical tools for detecting and measuring various analytes. In combination with flow cytometry systems, microspheres and their associated analytes may be separated and analyzed. Microspheres are also referred to in the art as polymeric beads, particles, microbeads, and micro particles. Modern flow cytometers are able to analyze several thousand particles per second, and can actively separate and isolate such particles according to their specified properties based on the detection mechanism utilized. Through the careful selection of the types and sizes of microspheres, as well as the application of specific antibodies, lectins, and other molecules capable of binding to microspheres, flow cytometers may be used to analyze analytes contained in a small amount of biological sample.

### SUMMARY OF THE DISCLOSURE

It is, therefore, a principle object of the subject invention to provide a method of using antibodies attached to different types of microspheres against different antigens located within a biological sample. Both optical and electronic particle detection are used to separate the microspheres via flow cytometry. This novel method will allow the measurement of multiple analytes in a single sample of body fluid, with each analyte separated by gating based on the type of microsphere to which the analyte is coupled. According to the present disclosure, various biological components are attached to microspheres of different volumes, shapes, conductivity, densities, and/or colors to detect biological components by gating on the type of microsphere and analyzing the biological component attached thereto.

Applicant has addressed the need for faster, more reliable measurement of multiple analytes, requiring minimal sample sizes by providing a method for using electronic particle volume to differentiate microspheres and their associated analytes. Additional objects and advantages of the disclosure are set forth in, or will be apparent to those of ordinary skill in the art from, the detailed description as follows. Also, it should be further appreciated that modifications and variations to the specifically illustrated and discussed methods and compositions hereof may be practiced in various embodiments and uses of this invention without departing from the spirit and scope thereof, by virtue of present reference thereto. Such variations may include, but are not limited to, substitutions of the equivalent means, features, and compositions for those shown or discussed, and the functional or positional reversal of various parts, features, method steps, or the like.

Still further, it is to be understood that different embodiments, as well as different presently preferred embodiments, of this invention may include various combinations or configurations of presently disclosed features, elements, method steps, or their equivalents, including combinations of features or configurations thereof not expressly stated in the detailed description. These and other features, aspects, and advantages of the present invention will become better understood with reference to the following descriptions and the appended claims.

### BRIEF DESCRIPTION OF THE DRAWINGS

FIG. 1 depicts various antibodies attached to microspheres of different sizes.

FIG. 2 is a flowchart depicting the exemplary method of using flow cytometry to measure multiple analytes.

### DETAILED DESCRIPTION

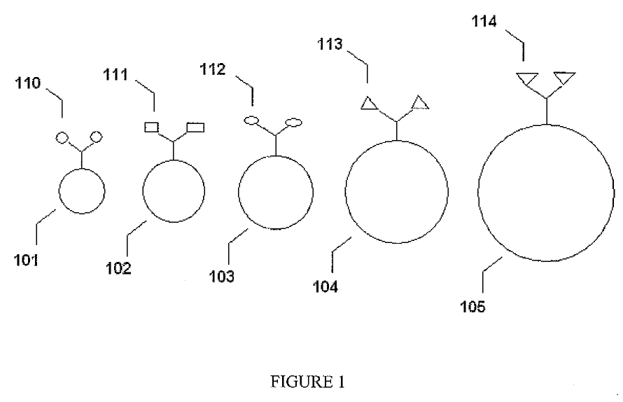

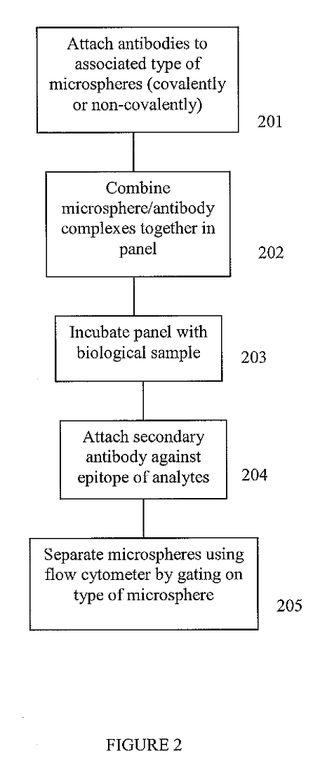

The various embodiments of the present disclosure and their advantages are best understood by referring to FIGS. 1 and 2 of the drawings. It will be apparent to those skilled in the art that various modifications and variations can be made in the present disclosure without departing from the scope and spirit of the disclosure as described herein. For instance, features illustrated or described as part of one embodiment can be included in another embodiment to yield a still further embodiment. Moreover, variations in selection of materials and/or characteristics may be practiced to satisfy particular desired user criteria. Thus, it is intended that the present disclosure covers such modifications as come within the scope of the present features and their equivalents.

According to the present disclosure, antibodies specific to known antigens are attached to corresponding microspheres of different types, wherein each antibody is associated with a different type of microsphere. In an alternative embodiment, any particle capable of binding analytes may be used and attached to the microspheres.

The types of antibodies utilized will depend upon the predicted analytes, or the analytes under investigation. The type of microsphere used will depend upon the number of antibodies utilized. For example, if the investigator is examining five (5) analytes in a given biological sample, he or she may use five (5) different antibodies attached to microspheres of five (5) different diameters (2 microns, 4 microns, 6 microns, 8 microns, or 10 microns). As shown in FIG. 1, one antibody 110 is attached to the two (2) micron microsphere 101, another antibody 111 is attached to the four (4) micron microsphere 102, another antibody 112 is attached to the six (6) micron microsphere 103, another antibody 113 is attached to the eight (8) micron microsphere 104, and another antibody 114 is attached to the ten (10) micron microsphere 105. In another embodiment, where numerous analytes are under investigation, different types of microspheres may be differentiated for gating purposes using other parameters such as fluorescence, color, conductivity and density. In other words, different subsets of two micron microspheres may be separated from each other according to their color or fluorescence. While this example contemplates using microsphere diameters varying in size by two (2) microns, differentiating microspheres by as little as three tenths (0.3) microns in diameter is possible using flow cytometers known in the art.

As for the antibodies, in one example, one microsphere may have an angiostatin antibody or fragment thereof attached to the microspheres of a two (2) micron diameter with the Fc (fragment, crystalline) portion attached to the microsphere and the Fab (fragment, antigen-binding) portion exposed to bind angiostatin in a small sample of blood. After incubation and washing, if necessary, the two (2) micron microspheres are incubated with a fluorescent labeled antibody against a different epitope of angiostatin. After washing, if necessary, the flow cytometer is gated on the microspheres by electronic particle volume and analyzed by fluorescence for the Angiostatin.

The method of the present disclosure is accomplished in several steps as depicted in FIG. 2. Each antibody type is attached to its associated microsphere by type of microsphere (size, fluorescence, color, conductivity, density) either covalently (e.g., malemide binding via sulphur groups) or non-covalently by simple adhesion (one hr at 4 C), as depicted in step 201. Next, the microsphere/antibody complexes are combined together in a panel, as depicted in step 202. Next, the panel of microsphere/antibody complexes is incubated with the biological samples for attachment of the analytes to the associated microsphere/antibody complex, as depicted in step 203. Next, each specific analyte is detected by attaching a unique second fluorescent-tagged antibody against a unique epitope of each analyte, as depicted in step 204. This second antibody is used to detect and quantify the analyte for each type of microsphere. Other means besides fluorescence can be used to detect the specific analyte, such as conductivity and absorbance. Finally, a flow cytometer known in the art, preferably one equipped with electron particle detection, will be used to separate the microspheres and their associated analytes by an electronic particle volume detection device, as depicted in step 205.

In an alternative embodiment where fluorescent microspheres are used, the flow cytometer incorporates optical detection and generates fluorescent emission spectra corresponding to the fluorescent microsphere. The detection device identifies the different types of microspheres by a unique combination of fluorescent labels and electronic particle volume gating, thereby separating microspheres based on size and fluorescence. Information from the fluorescent labels and the electronic particle volume analysis allows for a quantitative identification of the analytes in the sample. Software known in the art collects and analyzes the data generated by each microsphere and causes the device to separate them based upon type. After separation, each type of microsphere may then be analyzed to determine the associated analyte thereon using standard techniques such as fluorescence, ELISA assays, mass spectrometry, luminescence, light scatter and conductivity.

For purposes of this disclosure, the term analyte includes but is not limited to both organic and inorganic molecules capable of binding antibodies, such as proteins, ligands, carbohydrates, DNA, RNA, peptides, angiostatins, endostatins, trombospotins, ceruloplasim, cytokeratins, VEGF, angiogenic markers, Reactive Oxidation Species, Natural Killer Cell (NKC) markers. The present disclosure may be employed in detecting analytes for use in serology, diagnostic assays, as well as other assays such as therapeutic drug administration, monitoring and research, as well as oligonucleotide assays.

An advantage of the invention is that multiple analytes may be detected simultaneously. Each sub-population of microspheres may be tagged with antibodies specific to different analytes of interest. A panel of microspheres is allowed to react with a sample and then pass through the detection system where the analytes may be simultaneously detected and quantified. Data from the known class of microspheres can be compared to data detected from the sample microspheres of an unknown class.

It will be apparent to those skilled in the art that various modifications and variations can be made in the present disclosure without departing from the scope and spirit of the disclosure as described herein. For instance, features illustrated or described as part of one embodiment can be included in another embodiment to yield a still further embodiment. Moreover, variations in selection of materials and/or characteristics may be practiced to satisfy particular desired user criteria. Thus, it is intended that the present disclosure covers such modifications as come within the scope of the present features and their equivalents.

Although an embodiment of the disclosure has been described using specific terms and devices, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of various other embodiments may be interchanged both in whole or in part. Therefore, the spirit and scope of the appended claims should not be limited to the description of the preferred version contained herein.

I claim:

1. A method of detecting, measuring, and analyzing a plurality of analytes in a biological sample, said method comprising:
a. attaching a subset of microspheres to a corresponding subset of antibodies specific to one of said analytes;
b. repeating step (a) until each subset of microspheres has been attached to a subset of antibodies specific to each of said analytes;
c. combining said microspheres into a panel;
d. mixing said panel of microspheres with said sample;
e. attaching a second labeled antibody for detection of said analytes.
f. utilizing a flow cytometer to separate each subset of microspheres according to a predefined property of said microsphere;

2. The method of claim 1 wherein said predefined property of the microsphere is selected from one of the group consisting of size, fluorescence, color, density, or conductivity.

3. The method of claim 1 wherein said separation is accomplished by gating on said predefined property.

4. The method of claim 1 wherein said first attaching step is accomplished through covalent bonding.

5. The method of claim 1 wherein said first attaching step is accomplished through non-covalent bonding.

6. The method of claim 1 wherein said analyte is selected from one of the group consisting of an antibody, antibody fragment, ligand, carbohydrate, DNA, RNA, protein, peptide, angiostatin, endostatin, trombospotin 1, vascular endothelial growth factor, angiogenic marker, antiangiogenic marker, Reactive Oxidation Species, Natural Killer Particles, bacterium, virus or cell.

7. The method of claim 1 further comprising the step of analyzing each subset of microspheres for particular analytes.

8. The method of claim 1 wherein said second labeled antibody is labeled with a fluorescent tag.

9. A method of analyzing a plurality of analytes in a biological sample, said method comprising:
a. attaching a subset of microspheres to a corresponding subset of antibodies specific to one of said analytes;
b. attaching a subset of labeled antibodies to an epitope region on said analytes;
c. repeating steps (a) and (b) until each subset of microspheres has been attached to a subset of antibodies specific to each of said analytes,
d. combining said microspheres into a panel;
e. mixing said panel of microspheres with said sample;
f. utilizing a flow cytometer to separate each subset of microspheres according to a predefined property of said microsphere;

10. The method of claim 9 wherein said predefined property of the microsphere is selected from the group consisting of size, fluorescence, color, density, conductivity.

11. The method of claim 9 wherein said separation is accomplished by gating on said predefined property.

12. The method of claim 9 wherein said first attaching step is accomplished through covalent bonding.

13. The method of claim 9 wherein said first attaching step is accomplished through non-covalent bonding.

14. The method of claim 9 wherein said analyte is selected from one of the group consisting of an antibody, antibody fragment, ligand, carbohydrate, DNA, RNA, protein, peptide, angiostatin, endostatin, trombospotin 1, vascular endothelial growth factor, angiogenic marker, antiangiogenic marker, Reactive Oxidation Species, Natural Killer Particles, bacterium, virus or cell.

15. The method of claim 9 further comprising the step of analyzing each subset of microspheres for particular analytes.

16. The method of claim 9 wherein said subset of labeled antibody are labeled with fluorescent tags.

17. A method of detecting, measuring, and analyzing a plurality of analytes in a biological sample, said method comprising:
a. attaching a subset of microspheres to a corresponding subset of particles specific to one of said analytes;
b. repeating step (a) until each subset of microspheres has been attached to a subset of particles specific to each of said analytes;
c. combining said microspheres into a panel;
d. mixing said panel of microspheres with said sample;
e. attaching a second labeled antibody for detection of said analytes.
f. utilizing a flow cytometer to separate each subset of microspheres according to a predefined property of said microsphere;

18. The method of claim 17 wherein said predefined property of the microsphere is selected from one of the group consisting of size, fluorescence, color, density, or conductivity.

19. The method of claim 17 wherein said separation is accomplished by gating on said predefined property.

20. The method of claim 17 wherein said first attaching step is accomplished through covalent bonding.

21. The method of claim 17 wherein said first attaching step is accomplished through non-covalent bonding.

22. The method of claim 17 wherein said analyte is selected from one of the group consisting of an antibody, antibody fragment, ligand, carbohydrate, DNA, RNA, protein, peptide, angiostatin, endostatin, trombospotin 1, vascular endothelial growth factor, angiogenic marker, antiangiogenic marker, Reactive Oxidation Species, Natural Killer Particles, bacterium, virus or cell.

23. The method of claim 17 further comprising the step of analyzing each subset of microspheres for particular analytes.

24. The method of claim 17 wherein said second labeled antibody is labeled with a fluorescent tag.
